(19) United States
(12) Patent Application Publication
(10) Pub. No.: US 2012/0094348 A1
(43) Pub. Date: Apr. 19, 2012

(54) ORGANOSOLV BIOREFINING OF WHOLE SUGAR CANE

(76) Inventors: Edward Kendall Pye, Burnaby (CA); Michael Rushton, Burnaby (CA); John Ross Maclachlan, Burnaby (CA)

(21) Appl. No.: 13/146,399

(22) PCT Filed: Jan. 15, 2010
(86) PCT No.: PCT/CA10/00057

§ 371 Date: Dec. 14, 2011

Related U.S. Application Data
(60) Provisional application No. 61/145,478, filed on Jan. 16, 2009.

(51) Int. Cl. C12P 7/14; C13K 13/00; C12M 1/40

(52) U.S. Cl. 435/162; 435/289.1; 127/1

(57) ABSTRACT

An apparatus for processing sugar cane to concurrently produce sugar from cane juice, and ethanol and other co-products from bagasse. The apparatus comprises equipment for separating a cane juice stream and a fibrous bagasse from a sugar cane feed-stock, equipment for refining the cane juice, equipment for processing the fibrous bagasse for recovery therefrom of a cellulosic pulp and a liquor stream, equipment for saccharification and fermentation of the cellulosic pulp to produce a fermentation beer therefrom, and equipment for recovery of an ethanol stream from the fermentation beer. Legacy sugar mills may be retrofitted with a bagasse biorefining apparatus to concurrently produce ethanol and co-products, with existing cane juice extraction and processing operations.

### FIELD OF THE INVENTION

This invention relates to concurrent production of sugar streams and cellulosic ethanol from whole sugar cane. More particularly, this invention relates to equipment, systems and processes for biorefining of whole sugar cane to produce sugar streams, cellulosic ethanol and other co-products.

### BACKGROUND OF THE INVENTION

Sugar cane, a C₄ photosynthetic plant, has one of the highest productivities and yields per acre of any plant when grown under tropical conditions. It has been grown for centuries for the production of table sugar and molasses from which rum is produced. The primary process for producing sugar was to extract juice from freshly cut sugar cane and then to concentrate the juice by evaporation until the sugar crystallized and could be recovered by filtration as raw sugar. In the early days of the industry, many sugar mills consisted of wind-powered, heavy twin rollers into which the cane stalks were fed at the nip. The cane was thus crushed and the expressed sugar juice was collected for further processing. The residual crushed cane solids, known as bagasse, still contained major quantities of sugar since the bagasse could only be squeezed to about 50% solids. This represented significant losses of sugar yield. In the 19^{th} century, this loss of yield was addressed by adding expensive counter-current washing systems for post-crushing processing of the bagasse. These high capital cost innovations increased the yield of sugar juice but required more energy for the evaporation of the added wash water, which in turn led to the introduction of more efficient evaporators, such as multiple effect evaporators, which also increased the capital cost and technical complexity of the mills. The final wet, residual bagasse, was rejected from the mill and stored outside in huge piles. Most sugar mills burn the bagasse as a low-value fuel to provide the steam and power are needed for evaporation of the diluted cane juice, but, with modern evaporators and other efficiency improvements, modern mills do not need all the bagasse from the harvest. Consequently, bagasse is stored in giant piles and left to rot thereby producing negative environmental consequences with no economic returns to the mills. Production of sugar cane increased dramatically in recent years, particularly in South America, for use in production of fuel ethanol further increasing the accumulations of bagasse.

### SUMMARY OF THE INVENTION

The exemplary embodiments of the present invention relate apparatus, systems and process for biorefining of sugar cane for concurrent production of sugar streams from cane juice, and cellulosic ethanol, and other co-products from fibrous bagasse waste materials.

Some exemplary embodiments of the present invention relate to bagasse biorefining apparatus for receiving and processing bagasse waste materials concurrent with cane juice extraction. The bagasse waste materials are pulped to produce cellulosic materials which are then saccharified, i.e., hydrolyzed to produce sugar streams. The sugar streams are fermented to produce fermentation beers that are subsequently distilled for recovery of ethanol. The ethanol may be further refined and processed into fuel-grade ethanols. The bagasse biorefining apparatus may be included in the design and construction of new sugar mill installations. The bagasse biorefining apparatus may also be retrofitted to legacy sugar mill operations. The bagasse biorefining apparatus may be used to reduce bagasse stockpile accumulations at the end of annual sugar production cycles.

The bagasse waste materials are pulped by organosolv processes wherein the bagasse materials are pulped by commingling with suitable organic solvents in suitable heated and pressurized vessels. Suitable organic solvents are exemplified by short-chain alcohols, organic acids, ketones, and mixtures. Short-chain alcohols exemplified by methanol, ethanol, butanol, and propanol are particularly useful for organosolv pulping of bagasse. Cellulosic pulps produced from bagasse by organosolv pulping are separated from black liquors comprising spent solvents and solubilized and/or fractionated components. The cellulosic pulps are transferred to equipment for enzymatic hydrolysis to produce sugar streams. The sugar streams may then be transferred to fermenters for culture with suitable fermentative microrganisms to produce beers. It is optional to transfer the cellulosic pulp into equipment configured for concurrent saccharification and fermentation in the same vessel, i.e., in CSF vessels. The beers are distilled in distillation towers for separation and recovery of ethanol and stillage. The stillage may be disposed or alternatively, used to adjust the viscosity of the cellulosic pulps prior to enzymatic hydrolysis.

Equipment may be provided for recovery of spent solvents from black liquors. The spent solvents may then recharged by mixing with fresh solvents, and recycled for additional organosolv pulping. The black liquors may first be de-lignified prior to solvent recovery. Novel lignin derivatives may be recovered during de-lignification, and used for other industrial applications. Equipment may also be provided for recovery of furfurals and for processing stillages separated from the spent solvent for recovery of other organic compounds exemplified by acetic acid, formic acid, sugar syrups that may comprise one or more hexoses and pentoses. The apparatus and equipment may be configured do deliver a portion of the recovered sugar syrups to the fermentation equipment for increased yields of ethanol from the bagasse waste materials. The saccharification and fermentation equipment may also be configured to receive inputs from cane juice streams, sugar streams and molasses streams.

Other exemplary embodiments of the present invention relate to modifications to continuous countercurrent vertical extractors originally configured for organosolv processing of lignocellulosic biomass feedstocks, to make the vertical extractors suitable for receiving therein sugar cane, billets, pressing cane juice therefrom, and separating the cane juice from the sugar cane fibres, i.e., the bagasse.

### BRIEF DESCRIPTION OF THE DRAWINGS

The present invention is described in conjunction with reference to the following drawings, in which:

FIG. 1 is a schematic flowchart of an exemplary embodiment of the present invention for biorefining of whole sugar cane to concurrently produce a sugar stream and cellulosic ethanol;

FIG. 2 is an expanded schematic flowchart of the embodiment from FIG. 1, showing additional steps for recovery of extractives and other coproducts from organsolv processing of sugar cane bagasse;

FIG. 3 is a schematic flowchart of an exemplary embodiment of the present invention for retrofitting an operational sugar mill with organosolv biorefining system for concurrent production of a sugar stream and cellulosic ethanol;

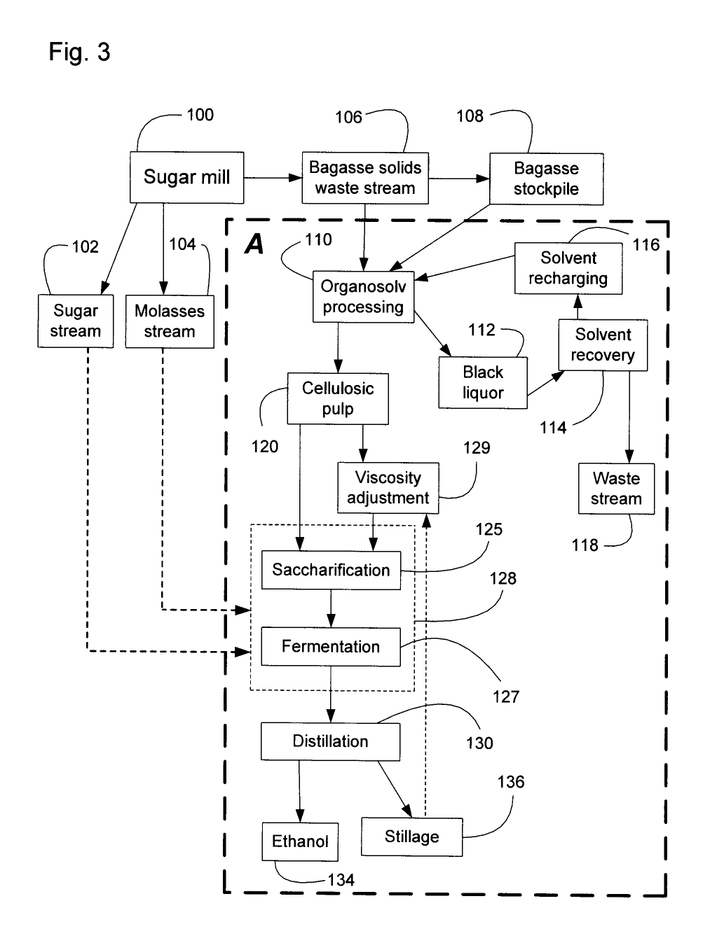

FIG. 4 is an expanded schematic flowchart of the embodiment from FIG. 3, showing additional components that may be added for recovery of extractives and other coproducts from organsolv processing of sugar cane bagasse; and

FIG. 5 is a schematic flowchart showing the configuration of an exemplary screw-press configuration multiple sequential extraction of cane juice from sugar cane billets and bagasse.

### DETAILED DESCRIPTION OF THE INVENTION

The exemplary embodiments of the present invention relate to biorefinery systems and integrated processes for processing whole sugar cane to concurrently produce: (a) a raw sugar stream and bagasse solids materials, and (b) cellulosic ethanol from the bagasse materials.

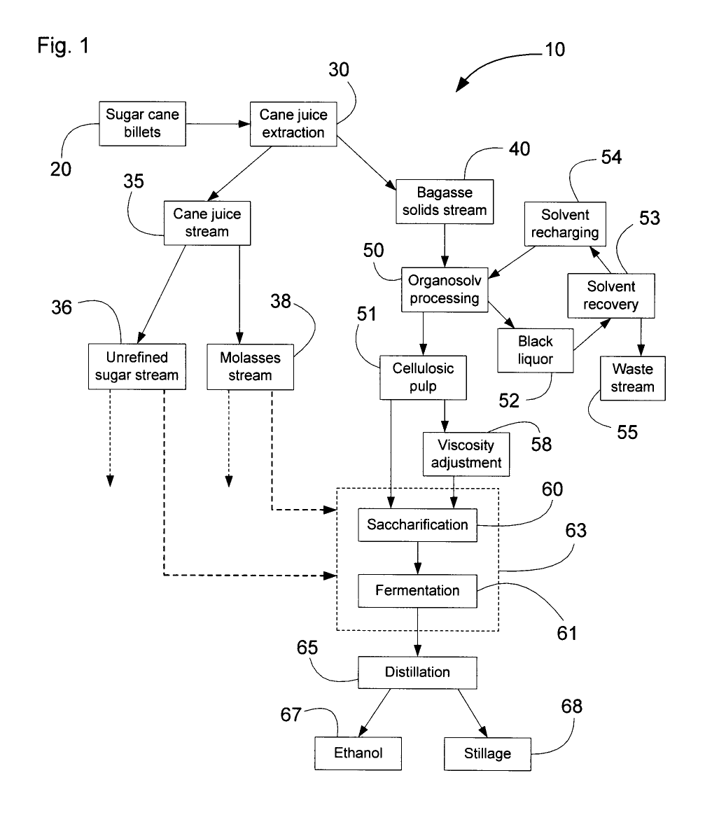

FIG. 1 shows an exemplary whole sugar cane processing system 10 for concurrently producing an unrefined sugar stream and ethanol according to an embodiment of the present invention. Sugar cane billets 20 are fed into cane juice extraction equipment 30 for crushing, washing and separation into a cane juice stream 35 and a bagasse solids stream 40. The cane juice stream 35 may be separated into an unrefined sugar stream 36 and a molasses stream 38 which are separately collected for further processing. The bagasse solids stream 40 is transferred into suitable organosolv processing equipment 50 wherein it is commingled with a suitable organic solvent selected for organosolv processing of the bagasse solids to separate therefrom cellulosic pulp materials 51. The cellulosic pulp materials 51 are transferred into saccharification equipment 60 wherein they are enzymatically hydrolysed to produce a monosaccharides stream. Suitable enzyme preparations for hydrolysis of cellulosic pulp materials into hemicelluloses, polysaccharides, oligosaccharides and monosaccharides may comprise one or more of enzymes exemplified by endo-β-1,4-glucanases, cellobiohydrolases, β-glucosidases, β-xylosidases, xylanases, α-amylases, β-amylases, pullulases, esterases, other hemicellulases and cellulases and the like. However, prior to delivery of the cellulosic pulp materials 51 to the saccharification equipment 60, it is optional to transfer some or all of the cellulosic materials 51 to equipment 58 wherein its viscosity can be controllably adjusted by intermixing with a suitable liquid exemplified by water or stillage 68 recovered from the distillation equiment 65, after which, the viscosity-adjusted cellulosic materials are transferred to the saccharification equipment 60 for enzymatic hydrolysis. The monosaccharides stream is then transferred from the saccharification equipment 60 to the fermentation equipment 61 and cultured with suitable fermentative microorganisms to produce a beer. Suitable microbial inocula for fermenting the monosaccharides stream may comprise one or more suitable strains selected from yeast species, fungal species and bacterial species. Suitable yeasts are exemplified by Saccharomyces spp. and Pichia spp. Suitable Saccharomyces spp are exemplified by S. cerevisiae such as strains Sc Y-1528, Tembec-1 and the like. Suitable fungal species are exemplified by Aspergillus spp. and Trichoderma spp. Suitable bacteria are exemplified by Escherichia coli, Zymomonas spp., Clostridium spp., and Corynebacterium spp. among others, naturally occurring and genetically modified. It is within the scope of the present invention to provide an inoculum comprising a single strain, or alternatively a plurality of strains from a single type of organism, or further alternatively, mixtures of strains comprising strains from multiple species and microbial types (i.e. yeasts, fungi and bacteria). The beer is transferred to distillation equipment 65 for recovery of ethanol 67 and stillage 68. The recovered ethanol 67 may be further refined and/or processed to make it suitable for use as a fuel ethanol. With regard to saccharication and fermentation of the sugar streams produced from the cellulosic pulp materials 51, the saccharification and fermentation steps can be optionally conducted concurrently in suitable equipment and systems known to those skilled in these arts as concurrent saccharification and fermentation equipment and processes 63. It is optional to divert a portion of the unrefined sugar stream 36 to the fermentation equipment 61 or alternatively to the concurrent saccharification and fermentation equipment 53. It is also optional to divert a portion of the molasses stream 38 to the fermentation equipment 61 or alternatively to the concurrent saccharification and fermentation equipment 63.

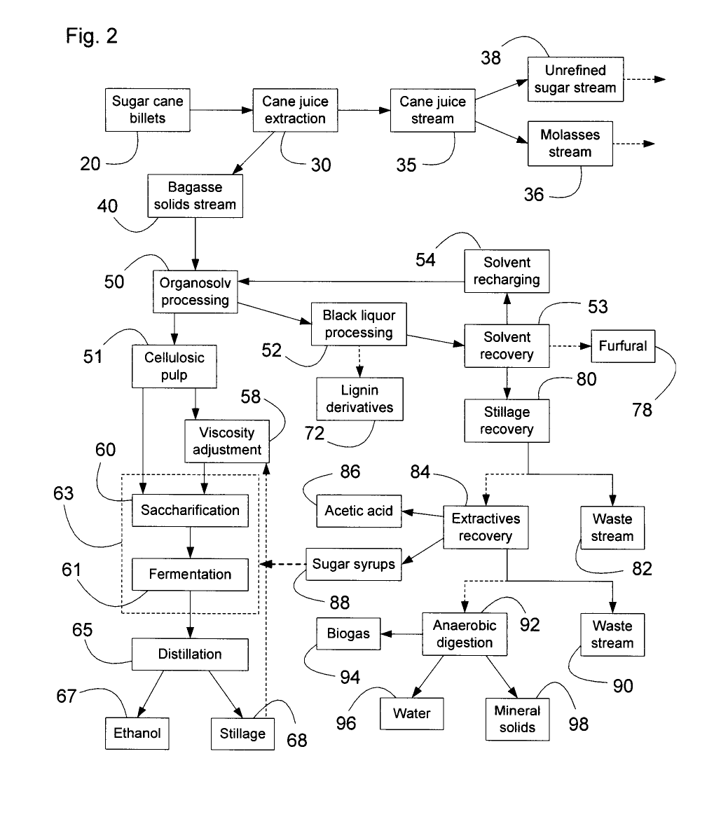

Suitable digestion/extraction equipment for organosolv processing of the bagasse are exemplified by counterflow or countercurrent digesters and concurrent flow digesters among others. In accordance with an exemplary embodiment of the present invention, the bagasse solids stream 40 is delivered into one end of a countercurrent digester and conveyed to the opposite end therein with a screw-type auger. Pressurized and heated organic solvent is delivered through an inlet at the end of the digester opposite to the bagasse input end and counterflows against the movement of the bagasse through the digester thereby providing turbulence and commingling of the solvent with the bagasse solids stream. Alternatively, the inlet for receiving the pressurized stream of heated digestion/extraction solvent may be provided about the bagasse input end of the digestion/extraction vessel or further alternatively, interposed the two ends of the digestion/extraction vessel. Exemplary organic solvents suitable for organosolv processing of bagasse solids streams include methanol, ethanol, propanol, butanol, acetone, and suitable mixtures thereof. If so desired, the organic solvents may be additionally controllably acidified by the addition of an inorganic or organic acid. If so desired, the pH of the organic solvents may be controllably manipulated by the addition of an inorganic or organic base. The commingling of the pressurized, heated organic solvent with the bagasse solids stream may be referred to as a cooking process. It is suitable for the digestion/extraction vessel to be controllably pressurized and temperature-controlled to enable manipulation of pressure and temperature so that target cooking conditions are provided while the organic solvent is commingling with the feedstock. Exemplary cooking conditions include pressures in the range of about 15-40 bar (g), temperatures in the range of about 120-350° C., and pHs in the range of about 0.5-5.5. During the cooking process, lignins and lignin-containing compounds contained within the bagasse solids stream will be fractionated and/or dissolved into the organic solvent resulting in the cellulosic fibrous materials previously adhered thereto and therewith to disassociate and to separate from each other. Those skilled in these arts will understand that in addition to the dissolution of lignins and lignin-containing polymers, the cooking process will release from the bagasse into the organic solvents in solute and particulate forms, monosaccharides, oligosaccharides and polysaccharides, organic acids such as acetic acid, formic acid and levulinic acids, and other organic compounds exemplified by furfural and 5-hydroxymethyl furfural (5-HMF) among others. Those skilled in these arts refer to such organic solvents containing lignins, lignin-containing compounds, monosaccharides, oligosaccharides, polysaccharides, hemicelluloses and other organic compounds extracted from lignocellulosic feedstocks such as bagasse, as “black liquors” or “spent liquors”. The disassociated cellulosic fibrous materials released from the bagasse solids are conveyed to the output end of the digestion/extraction vessel where they are discharged via a second auger feeder which compresses the cellulosic fibrous materials into a solids fraction, i.e., a pulp which is then conveyed to the saccarification equipment 60 or alternatively to the concurrent saccharification and fermentation equipment 63. As shown in FIGS. 1 and 2, the black liquors 52 are separately discharged as a liquid fraction from the organosolv processing equipment 50. Spent organic solvent 53 may be recovered from the black liquors by distillation, then recharged 54 and recycled for further organosolv processing of fresh incoming bagasse solids streams. Stillage produced during distallation recovery of the spent solvent, may be discharged in a waste stream 55. It is optional to recover furfural 78 from the spent organic solvent during distillation. It is optional to process black liquors with de-liginification equipment 72 for recovery of novel lignin derivatives prior to recovery of spent solvents with solvent recovery 53.

As shown in FIG. 2, some or all of the stillage waste stream 80 produced during processing of black liquors may be diverted to suitable equipment 84 for recovery therefrom of extractives that were separated from the bagasse solids 40 during organosolv processing 50. Alternatively, the recovered stillage may be discarded as a waste stream 82. Extractives recovered from stillage may include acetic acid 86, sugar syrups 88, formic acid and levulinic acid (not shown). It is optional to convey some or all of the recovered sugar syrups 88 to the concurrent saccharification and fermentation equipment 58 or alternatively to the fermentation equipment 57 to increase the yield of ethanol produced from the bagasse solids stream.

Another exemplary embodiment of the present invention relates to organosolv biorefining systems and methods configured for retrofitting sugar cane processing mills designed primarily for extraction of cane juice for production of sugar and molasses, for processing bagasse to produce ethanol. Retrofitting organosolv systems to sugar cane processing mills will enable processing of stored bagasse stockpiles to produce ethanol and optionally to recover co-products derived from bagasse such as lignin derivatives, furfurals, organic acids, sugar syrups, and other organic compounds. The advantage is that the bagasse is already accumulated in a central location and it has very little alternative value once the needs of the sugar mill for power and steam have been met through the combustion of bagasse. Therefore, this represents a major opportunity for the apparatus, systems and processes disclosed herein, which are scalable to match the annual throughputs of existing sugar mills. Depending on the geographic location of the cane fields, most cane harvesting only occurs over a six month period. Retrofitting organosolv biorefining systems to existing sugar mills enables concurrent organosolv processing of at least a portion of the bagasse produced during sugar production at cane harvest time, and continued processing of stockpiled excess bagasse after annual sugar production by the mills has been completed. Thus, bagasse from one six month harvesting period can to be accumulated and used over a twelve month period. This is not a problem because bagasse can be easily stored (and often is) for the six months between harvesting seasons. But this implies that the biorefinery would need to be economically attractive on a relatively small scale, because it would need to survive on only six months production of bagasse. Accordingly, it is within the scope of the present invention to scale the organosolv biorefining systems and equipment to enable year-round processing of bagasse generated from processing annual sugar cane harvests for sugar production. Thus, the exemplary embodiments of the present invention are well-suited to the utilization of bagasse from the legacy table sugar production countries and areas. Such countries could continue with their present practices and still sell their primary product into the table sugar market, while using excess bagasse to create additional value for their operations. Such a system has numerous advantages over existing operations. It simplifies and improves the efficiency of the entire sugar mill operation by eliminating the need for bagasse washers and reduces the energy requirements for evaporation. In a single mill operation, concentrated sugar juice is recovered and the resultant bagasse is processed by modified and/or adapted organosolv technology to produce cellulosic ethanol and/or other fermentation products, lignin and various co-products of the process. The yield of sugar in the juice would be lower than with conventional sugar mill processing, but the value of the “lost” sugar would be recovered in the enhanced ethanol production or derivatives, as described above. Furthermore, the sugar juice recovered from such an operation would be more concentrated than the juice from a traditional mill operation because it would not be diluted with wash water. This would lead to lower evaporation costs (i.e. lower steam demand), smaller evaporators and lower water demand by the mill. This in turn would lower the need for bagasse as a fuel, therefore leaving more of the bagasse for use as a feedstock for a modular biorefinery, assuming that a traditional mill would run side-by-side with the modular biorefinery

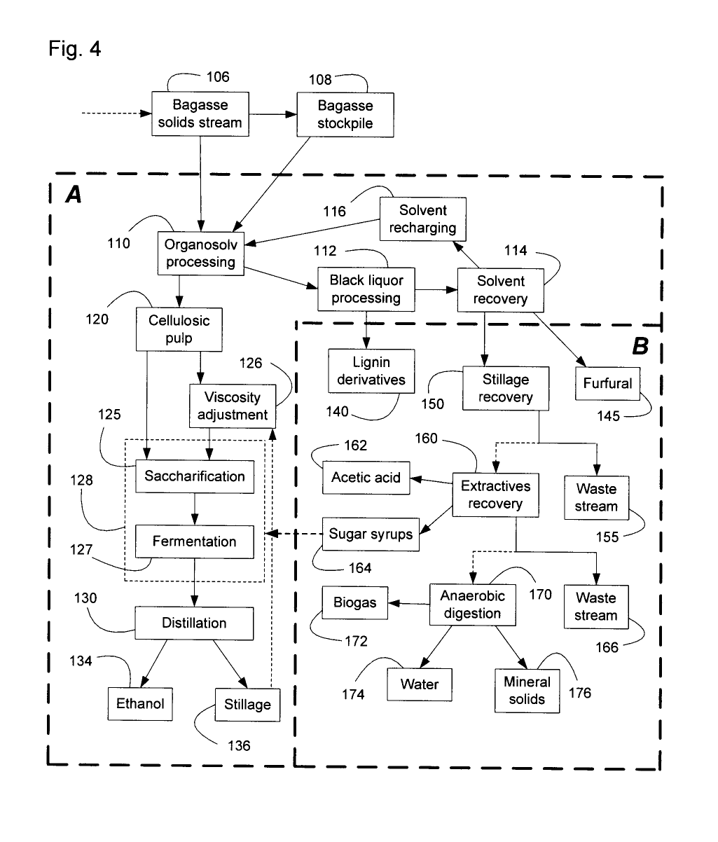

FIG. 4 is a flowchart of an exemplary legacy sugar mill 100 retrofitted with an organosolv biorefinery apparatus A (inside the hatched area) according to another embodiment of the present invention, for receiving and biorefining bagasse waste materials for production of ethanol. In its normal operation, the sugar mill 100 would process sugar cane billets to produce a sugar stream 102, a molasses stream 104, and a bagasse solids waste stream 106 that is stored in bagasse stockpiles 108. The sugar mill 100 is retrofitted with an organosolv biorefinery apparatus A that comprises organosolv equipment 110 exemplified by digesters for receiving and commingling bagasse with organic solvent there by producing cellulosic pulp 120 and black liquor 112. The organosolv biorefinery apparatus A also comprises equipment 114 exemplified by distillation towers for recovering spent solvent from the liquor and equipment for recharging the recovered solvent so that it can be recycled to the organosolv equipment 100 for use in further commingling with fresh bagasse inputs. The cellulosic pulp 120 is transferred to saccharification tank 125 for enzymatic hydrolysis to produce therefrom a monosaccharide sugar stream. A portion or alternatively, all of the cellulosic pulp 120 may be transferred into equipment 129 wherein the viscosity of the cellulosic pulp is adjusted to a target level. The viscosity-adjusted pulp is then transferred to the saccharification tank 125 for enzymatic hydrolysis. The monosaccharide sugar stream is transferred to a fermentation vessel 127 wherein it is cultured with fermentative microorganisms and converted into a beer. The beer is transferred to distillation equipment 130 exemplified by distillation towers for recovery of ethanol 134 and stillage 136. The ethanol 134 may be further processed and refined to make it suitable as a fuel ethanol. The stillage 136 may be recycled to the viscosity adjustment equipment 129 to adjust the viscosity of the cellulosic pulp 120 before it is delivered into the saccharification tank 125. It is optional for the cellulosic pulp 120 and viscosity-adjusted pulped to be transferred into a CSF tank 128 configured for concurrent saccharification and fermentation to produce a beer that is transferred to the distillation equipment 130. It is suitable to consider the organosolv biorefining apparatus A as a module for retrofitting an existing operating sugar mill to enable on-site production of ethanol from the bagasse waste material produced during sugar cane processing.

After a sugar mill has been upgraded with organosolv biorefining apparatus for ethanol production from bagasse, additional apparatus (FIG. 4, shown in hatched area B) may be retrofitted to the upgraded sugar mill at a later date(s) to enable further processing of black liquors for recovery therefrom of solubilized and particulate co-products that were fractionated and/or solubilized from bagasse solids waste material during commingling with organic solvents. As shown in FIG. 4, black liquor processing equipment may be installed for precipitation of solubilized lignin derivatives 140 prior to recovery of the spent organic solvent in distillation equipment 114. Suitable equipment 145 may be installed for capture and recovery of furfurals from distillation equipment 114 during recovery of spent organic solvents. Suitable equipment 150 can be installed for collection of stillage produced by distillation equipment 114 during recovery of the spent organic solvents. Suitable stillage processing equipment 160 can be installed for processing the stillage to recover therefrom organic acids exemplified by acetic acids 162 among others, and sugar syrups and other organic co-products. The apparatus provided for processing black liquors may be additionally configured to convey some or all of the stillage discharged from the solvent recovery equipment 114 through the stillage processing equipment 160, and may have controllable devices and instrumentation for diverting some or all of the stillage into a waste stream disposal systems 155 for discharge outside of the upgraded sugar mill. Anaerobic digestion equipment 170 may be interconnected with the waste stream disposal system 166 provided to receive the waste outputs from the stillage processing equipment 160, to receive and process at least some of the waste materials produced during recovery of organic acids and sugar syrups from stillage, for production therefrom of collectable biogas 172, water 174 and mineral solids 176. It is within the scope of the present invention to install and commission selected components of the additional apparatus on an as-needed or as-desired basis to extract more commercial value from the bagasse waste material. Alternatively, all of the components comprising the additional apparatus B may be installed at one time, ie. as a module to further upgrade a sugar mill that had been previously upgraded with an organosolv processing apparatus configured to produce ethanol from bagasse waste materials. Alternatively, a legacy sugar mill may be retrofitted with a system that comprises organosolv biorefining equipment configured for production of ethanaol from bagasse, and equipment for processing black liquors produced during organosolv biorefining of bagasse, to recover one or more of lignin derivatives, furfurals, organic acids, sugar syrups, organic phenolic compounds, biogas, and mineral solids.

Other exemplary embodiments of the present invention relate to modifications to continuous countercurrent vertical extractors originally configured for organosolv processing of lignocellulosic biomass feedstocks, to make the vertical extractors suitable for receiving therein sugar cane, billets, pressing cane juice therefrom, and separating the cane juice from the sugar cane fibres, i.e., the bagasse. A suitable exemplary continuous countercurrent extractor system comprises a plug-screw feeder configured to receive biomass at about the bottom of the extractor configured to receive a water supply about the top of the extractor, and to convey the biomass upwards to about the top of the extractor from where it egresses through a suitable conveyance device, e.g., a second plug-screw feeder provided therefor. The extractor is provided with at least an outer containment wall and a porous inner wall adjacent to the plug-screw feeder. The biomass, e.g., sugar cane billets, may be delivered directly to the bottom of the extractor. Alternatively, the sugar cane billets may conveyed from a holding container/bin to the bottom of the extractor with a separate plug-screw feeder configured to initiate the extraction of cane juice during its conveyance of the sugar cane biomass to a receptacle provided therefor at about the bottom of the vertical extractor. Plug-screw feeders force relatively moist materials such as sugar cane biomass, into high-density plugs thereby expressing liquids from the biomass. The expressed liquids are forced out through screens in the inner walls of the screw feeders. Those skilled in these arts will understand that the plurality of plug screw feeders provided with and cooperating with the exemplary continuous countercurrent vertical extractor serve the purposes of both crushing the cane biomass and squeezing out the cane juice in a similar manner as the roller mills employed in the prior art sugar mills. Those skilled in these arts will also understand that it is optional to configure the exemplary continuous countercurrent vertical extractor in a horizontal orientation or alternatively, in an angled orientation wherein the residual bagasse materials egress end is elevated relative to the biomass receiving end.

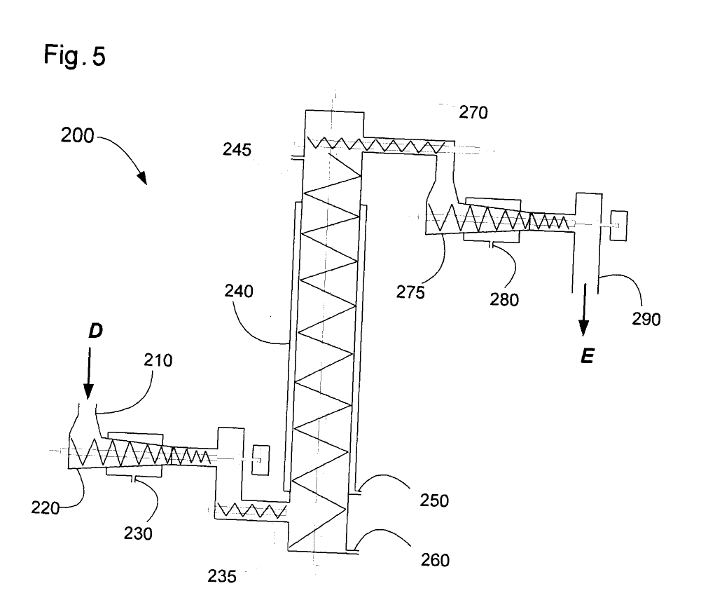

FIG. 5 shows an exemplary sugar cane processing equipment design according to an embodiment of the present invention. Sugar cane billets D are delivered into a first plug-screw feeder/extractor 220 wherein cane juice is expressed and collected from outlet 230 as the sugar cane billets are crushed and kneaded during conveyance to the outlet end of the first plug-screw feeder/extractor 220. Sugar cane bagasse outputs from the first plug-screw feeder/extractor 220 are transferred by an auger 235 to an input at about the bottom end of a vertical extractor 240. Additional cane juice is extracted from the bagasse by mechanical kneading and crushing as it is conveyed to the top of the vertical extractor 240. The cane juice is egressed from the outer containment and bottom areas of the vertical extractor 240 through outlets 250 and 260. It is suitable to provide multiple outlets for cane juice egress and collection from the vertical extractor 240. One or more water inlets 245 may be provided about the top portion of the vertical extractor 240 to facilitate washing of the bagasse and extraction of additional cane juice as the bagasse is conveyed to the top of the extractor. The bagasse is transferred by an auger 270 to a second plug-screw feeder/extractor 275 for additional extraction of cane juice from the bagasse. The cane juice is collected through outlet 280. The thrice-pressed bagasse output from the second plug-screw feeder/extractor 275 is transferred by a suitable conveyance system 290 for further processing, e.g., by organosolv biorefining E or to a bagasse storage pile.

What is claimed is:

1. An apparatus for processing sugar cane, the apparatus comprising:
equipment for separating a cane juice stream and a fibrous bagasse from a sugar cane feedstock;
equipment for processing the fibrous bagasse for recovery therefrom of a cellulosic pulp and a liquor stream;
equipment for saccharification and fermentation of the cellulosic pulp to produce a fermentation beer therefrom; and
equipment for recovery of an ethanol stream from the fermentation beer.

2. An apparatus according to claim 1, additionally comprising equipment for refining the cane juice stream.

3. An apparatus according to claim 1, wherein the fibrous bagasse processing equipment is configured for pulping of the fibrous bagasse with an organic solvent.

4. An apparatus according to claim 3, additionally comprising equipment for recovery and recycling of the organic solvent from the liquor stream.

5. An apparatus according to claim 3, additionally comprising equipment for recovery from the liquor stream of one of lignin derivatives, furfurals, organic acids, and sugar syrups.

6. An apparatus according to claim 5, additionally configured to convey a portion of the sugar syrups to the fermentation equipment.

7. An apparatus according to claim 2, additionally configured to convey to the equipment for saccharification and fermentation, a portion of outputs from the refining equipment.

8. An apparatus according to claim 1, additionally configured to convey to the equipment for saccharification and fermentation, a portion of the cane juice stream.

9. An apparatus according to claim 1, additionally provided with equipment for anaerobic digestion of one or more waste streams produced during processing of the fibrous bagasse and/or recovery of ethanol from the fermentation beer.

10. A bagasse biorefining apparatus for retrofitting a sugar mill, the bagasse biorefining apparatus comprising:
equipment for processing a fibrous bagasse feedstock from the sugar mill for recovery therefrom of a cellulosic pulp and a liquor stream;
equipment for saccharification and fermentation of the cellulosic pulp to produce a fermentation beer therefrom; and
equipment for recovery of an ethanol stream from the fermentation beer.

11. A bagasse biorefining apparatus according to claim 10, wherein the fibrous bagasse processing equipment is configured for pulping of the fibrous bagasse with an organic solvent.

12. A bagasse biorefining apparatus according to claim 11, additionally comprising equipment for recovery and recycling of the organic solvent from the liquor stream.

13. A bagasse biorefining apparatus according to claim 11, additionally comprising equipment for recovery from the liquor stream of one of lignin derivatives, furfurals, organic acids, and sugar syrups.

14. A bagasse biorefining apparatus according to claim 13, additionally configured to convey a portion of the sugar syrups to the fermentation equipment.

15. A bagasse biorefining apparatus according to claim 10, wherein the equipment for saccharification and fermentation is additionally configured for receiving therein from the sugar mill one of a cane juice stream, a sugar stream and a molasses stream.

16. An apparatus according to claim 10, additionally provided with equipment for anaerobic digestion of one or more waste streams produced during processing of the fibrous bagasse and/or recovery of ethanol from the fermentation beer.

17. A process for biorefining sugar cane, the process comprising the steps of:
separating a cane juice stream and a fibrous bagasse from a sugar cane feedstock;
processing the fibrous bagasse for recovery therefrom of a cellulosic pulp and a liquor stream;
saccharification and fermentation of the cellulosic pulp to produce a fermentation beer therefrom; and
recovery of an ethanol stream from the fermentation beer.

18. A process according to claim 17, additionally comprising steps for refining the cane juice stream.

19. A process according to claim 17, wherein the fibrous bagasse processing step comprises pulping of the fibrous bagasse with an aqueous organic solvent.

20. A process according to claim 19, wherein the organic solvent is further defined as a short-chain alcohol, an organic acid, a ketone, or mixtures thereof.

21. A process according to claim 20, wherein the organic solvent comprises at least one short-chain alcohol further defined as a methanol, an ethanol, a butanol, a propanol, or an aromatic alcohol.

22. A process according to claim 19, wherein the organic solven is provided with a catalyst further defined as an inorganic acid or an organic acid.

23. A process according to claim 19, additionally comprising steps for recovery and recycling of the organic solvent.

24. A process according to claim 17, additionally comprising steps for recovery of lignin derivatives from the liquor stream.

25. A process according to claim 17, additionally comprising steps for recovery from the liquor stream of one of furfurals, organic acids, and sugar syrups.

26. A process according to claim 17, additionally comprising a step for delivering a portion of the cane juice stream to the saccharification and fermentation step.

27. A process according to claim 18, additionally comprising a step for delivery a portion of the refined cane juice stream to the saccharification and fermentation step.

28. A sugar cane processing apparatus, comprising:
a first plug-screw feeder for receiving a sugar cane feedstock and separating therefrom a cane juice stream and a fibrous bagasse material, the first plug-screw feeder having a bagasse discharge outlet about one end and a cane juice discharge outlet;
a continuous countercurrent extractor having a receptacle about one end for receiving the fibrous bagasse material discharged from the first plug-screw feeder, an auger for conveying the fibrous bagasse material to about the opposite end, a bagasse discharge outlet about the opposite end, and at least one cane juice discharge outlet; and
a second plug-screw feeder for receiving the bagasse material discharged from the countercurrent extractor, the second plug-screw feeder having a bagasse discharge outlet about one end and a cane juice discharge outlet.

29. A sugar cane processing apparatus according to claim 28, wherein a conveying device is provided for conveying the fibrous bagasse material from the first plug-screw feeder to the receptacle of the continuous countercurrent extractor.

30. A sugar cane processing apparatus according to claim 28, wherein a conveying device is provided for conveying the fibrous bagasse material from the continuous coountercurrent extractor to the second plug-screw feeder.

31. A sugar cane processing apparatus according to claim 28, wherein the countercurrent extractor comprises a double-walled housing containing therein an auger extending from about the bagasse receptacle end to about the bagass discharge end, said double-walled housing comprising an inner wall and a outer containment wall.

32. A sugar cane processing apparatus according to claim 31, wherein a portion of the wall interposed the bagasse receptacle end and the bagasse discharge end, is porous.

33. A sugar cane processing apparatus according to claim 31, wherein the countercurrent extractor is provided with at least one cane juice discharge outlet about the bagasse receptacle end and at least one cane juice discharge outlet interposed the bagasse receptacle end and the bagasse discharge end.
